(19) United States
(12) Patent Application Publication
(10) Pub. No.: US 2018/0199967 A1
(43) Pub. Date: Jul. 19, 2018

(54) IMPROVED PROSTHESIS FOR OSTEOSYNTHESIS

(71) Applicant: DA VINCI S.R.L., Verona (IT)

(72) Inventors: ENRICO RUSSO, ROMA (IT); RAFFAELE RUSSO, VILLARICCA (IT)

(21) Appl. No.: 15/741,850

(22) PCT Filed: Jul. 6, 2015
(86) PCT No.: PCT/IT2015/000179

§ 371 Date: Jan. 4, 2018

(51) Int. Cl. A61B 17/80; A61B 17/72

(52) U.S. Cl. CPC A61B 17/8061; A61B 17/8095; A61B 17/8805; A61B 17/8085; A61B 17/72; A61B 17/8057

(57) ABSTRACT

The present invention concerns a prosthesis for osteosynthesis (1) of a bone segment, in particular humerus (O), comprising at least one anchoring element (2), supporting means (3), coupled to the bone tissue of said bone segment (O), and positioning means (4) of said supporting means (3) with respect to said anchoring element (2).

### THE PRESENT INVENTION RELATES TO AN IMPROVED PROSTHESIS FOR OSTEOSYNTHESIS

More specifically, the invention concerns a prosthesis of the above kind, studied and realized in particular for the repair, the assembly, and the restoration of articular fragments of proximal fractures of the humerus of the human in their most different types, but which can be used for any type of bone fracture, in which the reconstruction of the bone tissue in the joint part is necessary, whether they are “broken” fragments and located out of their seat.

In the following, the description will be directed to proximal humeral fractures in humans, but it is clear that the same should not be considered limited to this specific use.

As it is well known, currently there are different types of prosthesis or systems for the synthesis of humerus proximal bone fractures. The main objective of these prostheses and/or osteosynthesis means, in addition to pain reduction, is to allow the recovery of the morphology of the affected bone segment and therefore also to allow the recovery of the mobility of the shoulder and then of the humerus, the elbow and arm in an all-comprehensive way.

Generally, the prostheses according to the prior art provide for the replacement of the articular part of the segment concerned, total or partial of the consumed head of the humerus with a metal ball fixed to a suitable support, often consisting of a stem, which is inserted in the diaphysis of the humerus. These systems include the use of ancillary screws or cylindrical-tight block systems or cement.

Solutions of this type have several technical problems. In particular, the necessity of the removal of the epiphyseal bone fragments of the humerus, replaced with ball or a metal hemisphere, does not allow any recovery of the bone tissue.

In addition, it often occurs the phenomenon in which the stem element, which is inserted longitudinally along the humerus axis, with or without the acrylic cement “slides” with respect to the same humerus towards the distal part of the channel and is mobilized. Alternatively, the system can bring a lengthening of the humeral segment because the points of recognition and orientation are lost after having removed the fragments. This phenomenon, in particular, occurs because stem is fixed to the humerus along the inner walls of a diaphyseal channel hollowed by the surgeon. For osteoporotic tissues, therefore, said anchoring is often unstable.

The Applicant has already partly addressed these technical problems in the patent application n. WO2006126238A2, in which it is described a prosthesis having a stem insertable in the diaphyseal channel of the humerus, and a surface with a concave surface, intended to contain and keep together the fractured bone tissue.

Such a prosthesis is very functional only from a theoretical point of view, because it has been shown by the international literature that when the surgical assembly between the pieces of residual bone and the metal prosthesis is created, a very large number of complications are creates, such as the non-assembly or improper assembly of the fragments. Indeed, these prostheses do not allow optimum recovery of some types of bone fractures, such as, for example, the four fragments fractures and the three fragments fractures and the humeral head four parts fractures and dislocations.

In particular, all the prostheses of the prior art are not optimal for osteosynthesis and the repair of any type of humerus proximal fracture of the human, e.g. complex fractures associated with involvement lower than the head of the humerus.

The prosthesis according to the prior art are described in patent applications WO2006/126238A2, WO01/67988A2, WO02/067811A2 and GB2471290A.

The patent application WO2006/126238A2 describes a prosthesis with an anchoring element and support means, coupled to the bone tissue. However, the patent application does not allow to place any positioning system of said support means with respect to said anchoring element.

Patent application n. WO01/67988A2 describes an apparatus that removes and replaces the head of the humerus, destroys the bone, it does not save and restore the humerus and the physiology of the joint, being unable to help the surgeon to preserve the articular cartilage. Furthermore, it does not include anchoring elements and a support coupled to the bone tissue.

Patent application n. WO02/067811A2 asks to delete the bone and it does not restore the physiology of the joint (for example) of the humerus, being unable to help the surgeon to preserve the articular cartilage.

Patent application n. GB2471290A does not include any anchoring element capable of supporting internally the fragments of the humeral head, thus allowing also a vascularization and avoiding slips that only plates and screws cannot avoid. Patent application n. GB2471290A starts already assuming to remove the cartilage and to replace the humeral head with a metal head.

The present invention, however, is a system for the conservation of bone and cartilage. It is an osteosynthesis with endosteal concept of volumetric filling and stabilization with different ways. Thus, it does not change the articulation and saves the bone-tendon structures, restoring the physiology of the joint allowing a rapid recovery of the humerus.

In light of the above, it is, therefore, object of the present invention to propose a prosthesis that overcomes the limits of the prior art.

One object of the present invention is to maximize the recovery of the bone tissue of the humerus, allowing the vascularization, as this system is admirably invented as a endosteal load-bearing structure, where the surgeon is facilitated and helped to restore anatomically all the fragments by matching easily all articular fracture lines, which in the past were considered not rebuildable.

A further object of the present invention is to avoid the slipping of the humeral head, and of the large and small tuberosity bone fragments of the humeral head after osteosynthesis, by anchoring to the humerus. This complication occurs with a very high percentage as it is clear from international literature, both with traditional prostheses as well as with commonly used synthesis systems, being screwed plates or screwed intramedullary nails. However, none of these systems allows a stable reconstruction of all the fragments with the restoration of anatomy and thus with the revascularization, avoiding very high percentages of necrosis found after the use of other systems.

It is therefore a specific object of the present invention a prosthesis for osteosynthesis of a bone segment, in particular humerus, comprising at least one anchoring element, supporting means, coupled to the bone tissue of said bone segment, and positioning means of said supporting means with respect to said anchoring element.

Always according to the invention, said at least one anchoring element could have an internal cavity and one or more surfaces openings or holes, communicating with said internal cavity, said supporting means and/or said positioning means being insertable within said internal cavity.

Still according to the invention, said anchoring element could have the shape of a polyhedron.

Further according to the invention, said polyhedron could have two triangle-shaped bases.

Advantageously according to the invention, said bases could be isosceles triangle shaped, said bases having a vertex angle and a pair of equal angles, said bases having a plurality of oval holes, and in that said polyhedron has: two side edges, corresponding to said equal angles of said bases; an vertex edge corresponding to said vertex angle, lower than said two side edges; two side surfaces each one bounded by said vertex edge and by a respective side edge, said side surfaces having at least one passage hole, preferably having an oval form; and a side surface, corresponding to the side of said base opposite to said vertex angle, bounded by said side edges, said side surface being open.

Always according to the invention, said bases could be equal and/or said vertex angle is greater than 90°.

Still according to the invention, said anchoring element could be made of titanium and/or resorbable material and/or inert material and/or fabric resulting from treated bone.

Advantageously according to the invention, said supporting means could comprise a plate, being substantially “Y” shaped or bat wings shaped, with two upper ends, said plate being provided with a plurality of threaded holes and being positionable on the side surface of said bone segment, and said fixing means comprise one or more threaded screws, insertable into the holes of said plate, engaging with said holes by the respective thread or by fixing, at least one of said threaded screws inserting through said surface passage holes, by entering into said internal cavity.

Always according to the invention, said plate could comprise one or more anchoring hooks on each of said upper ends.

Still according to the invention, said plate could be made of deformable material, so that it can adapt to the surface of said bone segment, even in the installation phase, such as malleable titanium, or the like.

Further according to the invention, said supporting means could comprise a stem, inserted in said bone segment, in order to be placed through said anchoring element within said cavity, said stem having one or more grooves on the side surface and a fixing threaded hole, arranged at one end, and a removable fixing structure, comprising an inverted-“L” shaped element, having a first side and second side arranged at about 90°, said first side having a plurality of guiding side through holes and an through upper hole, said fixing structure being positionable so that said first side is arranged parallel to said bone segment, and said fixing means comprise one or more threaded screws insertable through said guiding side holes, so that they arrange in said grooves, appropriately oriented, and a further screw, inserted in said upper hole, so as to engage with said fixing threaded hole of said stem.

Advantageously according to the invention, said supporting means could comprise at least one rigid guide having a first and a second end and insertable through the lateral surface of said bone segment, and a flange fixed to said first end, said at least one rigid guide being insertable in the bone tissue, so that said second end is inserted through at least one of said passage holes of said anchoring element, so that at least one portion said at least one rigid guide is arranged in said internal cavity, and said positioning means comprise at least one threaded screw, engageable by screwing with said flange and with the bone tissue of said bone segment.

Always according to the invention, said at least one rigid guide could have a curved shape.

Still according to the invention, said at least one rigid guide could have an internal channel, provided with an inlet hole and an outlet hole.

Further according to the invention, said at least one rigid guide could comprise a valve.

Advantageously according to the invention, said rigid guide could have an open lateral portion.

Always according to the invention, said supporting means could comprise a stem, insertable into a channel of said diaphyseal bone segment and inserted and arranged through said anchoring element within said cavity, and said positioning means comprise at least one hook arranged at one end of said stem, said hook being intended to couple with said anchoring element.

Still according to the invention, said at least one hook could be coupled in a removable way by said stem.

Advantageously according to the invention, said prosthesys could comprise a guide wire, made of metal or deformable material, insertable through said stem and said anchoring element, so as to approach said stem to said anchoring element.

Further according to the invention, said prosthesys could comprise a prosthetic structure, in its turn comprising a further stem, insertable within said bone segment, in particular within the diaphysis of the humerus, having an upper fastening pin and an interlocking or threaded cavity and, on the side surface, a plurality of longitudinal reliefs, insertable within the bone tissue of said bone segment, a cup or head, intended to be arranged on the proximal epiphysis of said bone segment and comprising a coupling ring provided with a rotatably adjustable seat, and a body, for connecting said further rod and said hood or head, said body comprising an upper portion, capable of coupling with said seat of said coupling ring of said hood or head, and a lower portion, oriented at an opening angle with respect to said upper portion and forming with it a shaped profile having a recess, in which said anchoring element is intended to be inserted, the lower end of said lower portion being provided with a coupling, in which said fastening pin of said stem is insertable and can be coupled in a removable way, said supporting means, being removably coupled with said interlocking or threaded cavity.

The present invention will be now described, for illustrative but not limitative purposes, according to its preferred embodiments, with particular reference to the figures of the enclosed drawings, wherein:

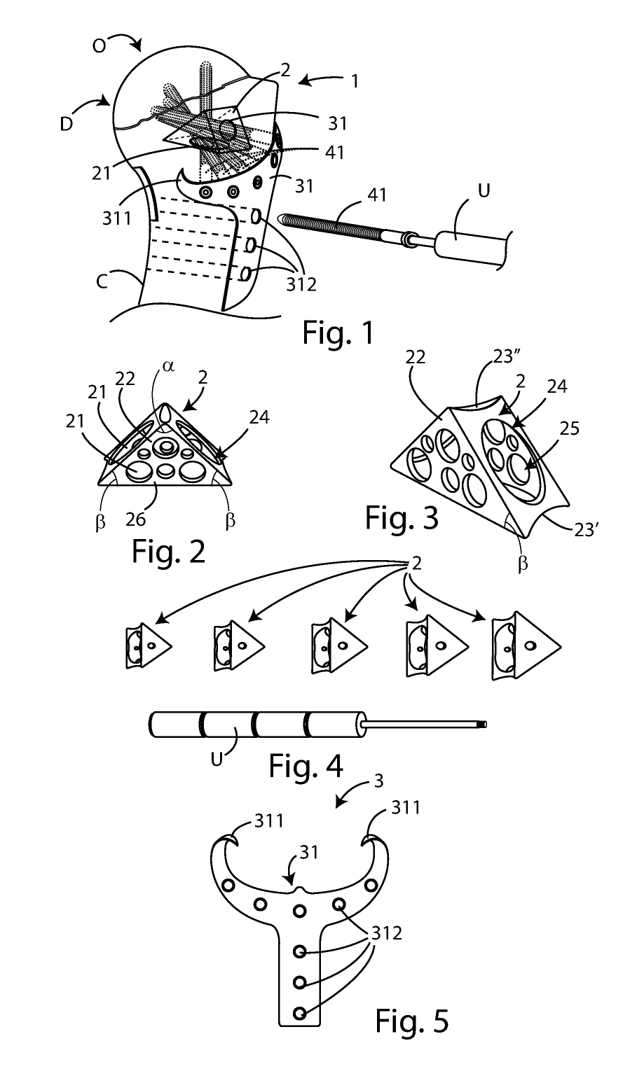

FIG. 1 shows a first embodiment of a prosthesis for osteosynthesis according to the present invention applied to a humerus;

FIG. 2 shows a perspective view of an anchoring element of the prosthesis for osteosynthesis according to FIG. 1;

FIG. 3 shows a further perspective view of the anchoring element of the prosthesis for osteosynthesis according to FIG. 2;

FIG. 4 shows a set of anchoring elements according to FIG. 2;

FIG. 5 shows a plate of the anchoring element of the prosthesis for osteosynthesis according to FIG. 1;

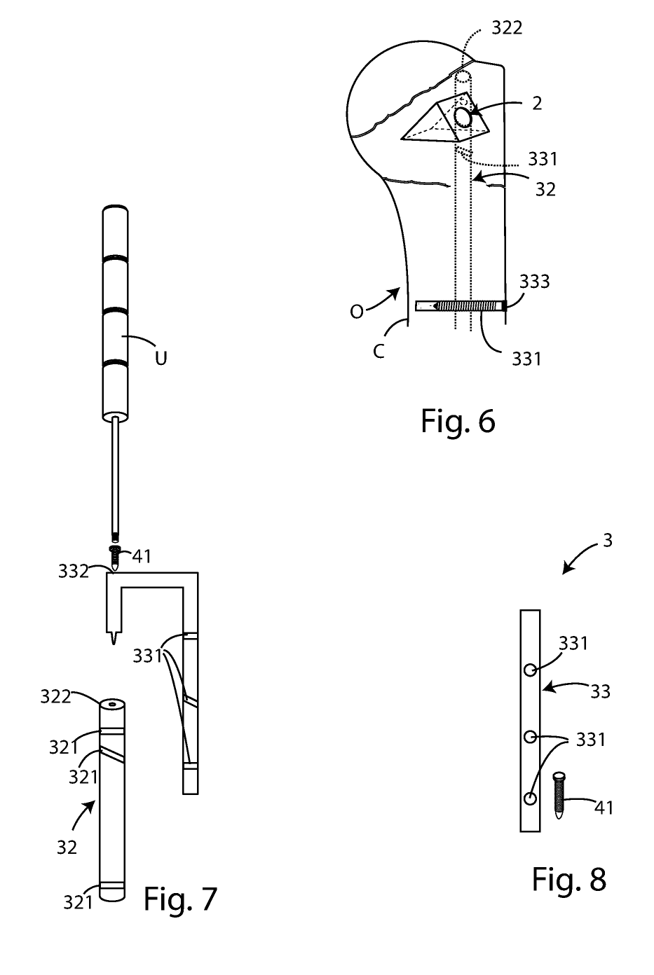

FIG. 6 shows a second embodiment of a prosthesys for osteosynthesis according to the present invention applied to a humerus;

FIG. 7 shows the support means of the prosthesis according to FIG. 6;

FIG. 8 shows a rear view of the fixing structure of the support means according to FIG. 7;

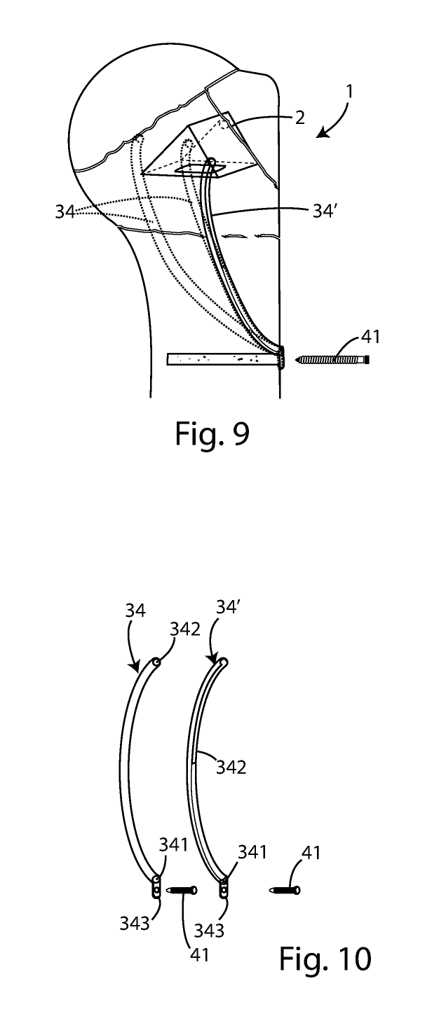

FIG. 9 shows a third embodiment of a prosthesis for osteosynthesis according to the present invention applied to a humerus;

FIG. 10 shows the rigid guides of the prosthesis of FIG. 9;

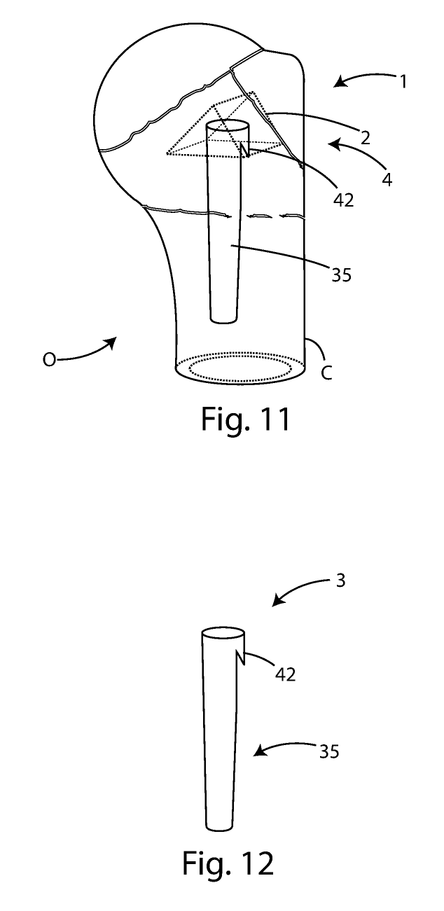

FIG. 11 shows a fourth embodiment of a prosthesis for osteosynthesis according to the present invention applied to a humerus;

FIG. 12 shows a perspective view of a stem of the prosthesis of FIG. 11;

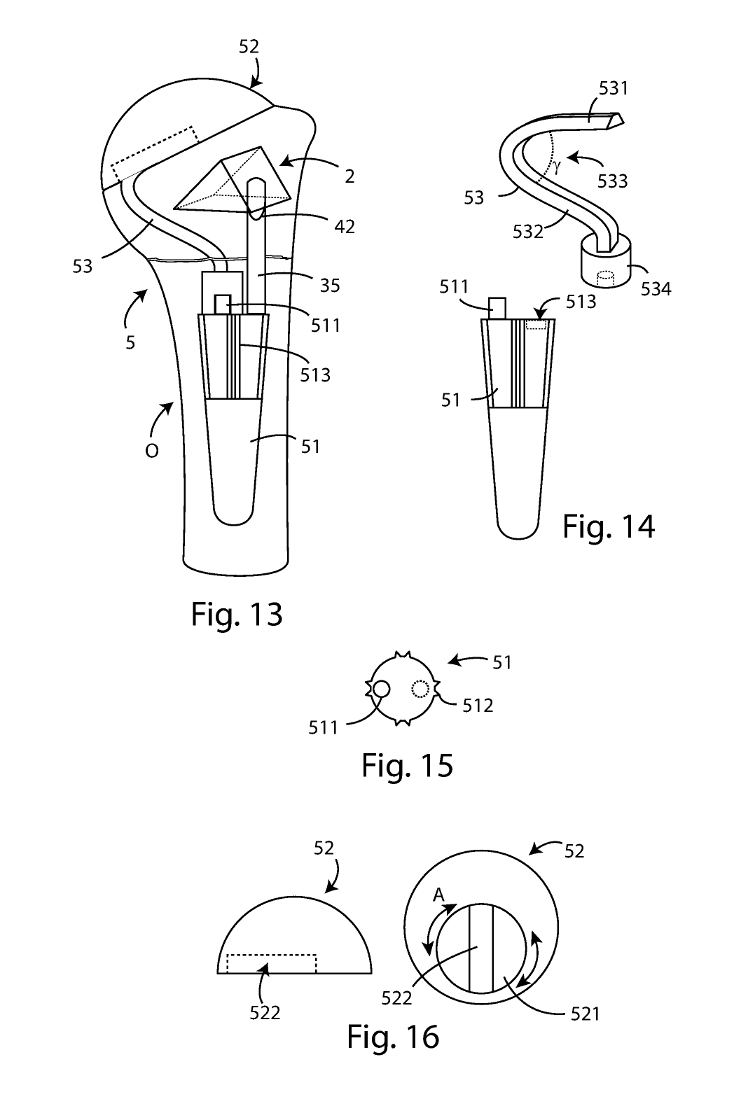

FIG. 13 shows the prosthesis for osteosynthesis according to FIG. 11 in combination with a prosthetic structure;

FIG. 14 shows an exploded view of the prosthetic structure of FIG. 13;

FIG. 15 shows the cross section of a further stem of the prosthetic structure according to FIG. 13; is

FIG. 16 shows the longitudinal section of a hood or head of the prosthetic structure according to FIG. 13.

In the various figures, similar parts will be indicated by the same reference numbers.

Referring to FIGS. 1-5, it is seen a first embodiment of the prosthesis for osteosynthesis according to the present invention, indicated by the reference number 1, which mainly includes an anchoring element 2, a support means 3 and positioning means 4, for maintaining said supporting means 3 in a suitable relative position with respect to said anchoring element 2.

In particular, the prosthesis for osteosynthesis 1 is applied to a humerus O, which, as it is known, is constituted by a body C, or diaphysis, and two ends, called epiphysis (proximal and distal). The proximal epiphysis P, shown in FIG. 1, articulates with the scapula, forming the shoulder joint, while the distal epiphysis (not shown in the figures) articulates with the two forearm bones, the radius and the ulna (not shown in the figures).

It should in any case be considered that the prosthesis for osteosynthesis 1 according to the invention, is applicable to any bone segment.

The prosthesis for osteosynthesis 1 applies between the proximal epiphysis P and the body C of the humerus O.

In particular, as it can be seen, the anchoring element 2 is internally hollow and has the shape of a polyhedron.

On the surfaces of said polyhedron, the anchoring element 2 has a plurality of openings or through holes 21, communicating with the internal cavity. The surfaces of said polyhedron identify, therefore, an internal cavity 24. Said through holes 21 have different sizes.

As it can be also observed in FIG. 4, said anchoring element 2 can have different sizes, to suit the different sizes of the humerus O (or of the bone segment in general) which is applied to. In particular, there are different types of measures such as 20×13 mm, 20×12 mm, 23×11 mm, 23×13, 25×11 mm, 25×13 mm, 28×11 mm, 28×13 mm, 30×11 mm and 30×13 mm.

In the present case the polyhedron has the bases 22 equal, shaping an isosceles triangle with vertex angle, in this case, a greater than 90°. The edges 23′, corresponding to the equal angles β of the bases 22 of the isosceles triangle, are of greater length of the edge 23″ corresponding to said vertex angle α.

The two lateral surfaces 25 of the anchoring element 2 corresponding to the equal sides of the isosceles triangle with base 22, have a passage hole 21′, having an oval shape. The surfaces of the bases 22 have a plurality of oval holes 21″ (in the case shown, six), of smaller dimensions of said oval hole 21′ and circular in shape. The lateral surface 26 of the anchoring element 2, corresponding to the side of said base 22, opposite to said angle α, is opened, but it could be also perforated.

The anchoring element 2 can also be realized in different ways, for example by means of bars welded together. Furthermore, the anchoring element 2 may have several possible shapes, according to the opportunity and the bone district in which it is applied to.

Furthermore, the anchoring element 2 can be made of different materials, such as, in particular, titanium, absorbable material or inert material.

Said anchoring element 2, due to its shape and variable sizes, is adaptable to various types of fractures, e.g. those in two parts or four parts of the humeral head P, for the synthesis and reconstruction.

The prosthesis for osteosynthesis 1 according to the invention can be applied alone or in combination with other systems, also belonging to the prior art.

The support means 3 in the present embodiment comprise a plate 31, being substantially “Y” or bat wings shaped, at the ends of the upper wings anchoring hooks 311 are obtained. Said plate 31 may also include a plurality of hooks 311.

Said plate 31 is provided on the surface of a plurality of threaded holes 312, whose function will be better explained in the following.

The positioning means 4, in this embodiment, are constituted by threaded screws 41.

The prosthesis for osteosynthesis 1 provides that the anchoring element 2 is intended to be inserted in the proximal epiphysis P, under the head of the humerus O, while the plate 31 is arranged on the side surface of the humerus O, as shown in FIG. 1.

Some of the threaded screws 41 are inserted, by means of a suitable tool U, in the holes 312 of the plate 31, engaging with said holes 312, by means of the respective thread with the bone tissue of the humerus O, thus anchoring the threaded screw 41 and, with it, the plaque to parts not fractured of the humerus O. Where appropriate, in a possible embodiment, the plate may not be threaded and the head of the threaded screw 41 corresponding is fixed to the same.

Other threaded screws 41, however, in addition to engage by means of the thread with the holes 312 of the plate 3, are inserted through the passage holes 21 of the anchoring element 2.

The anchoring element 2, being hollow, allows the vascularization of the internal cavity 24, so that the same can be filled with bone tissue, staring further threaded screws 41.

FIGS. 6 and 7 show a second embodiment of the prosthesis for osteosynthesis 1 according to the present invention.

In this case, the support means 3 comprise a stem 32 and a fixing structure 33 of said rod 32. Said rod 32 is insertable in a diaphyseal channel hollowed by the surgeon in the body of the humerus O and has grooves 321 on the side surface (which function will be better defined in the following), as well as a fixing threaded hole 322 at one end.

The fixing structure 33 comprises an inverted “L” shaped outer element, having a first side and a second side, arranged approximately at 90°. Said fixing structure 33 has a plurality of guiding side holes 331 on said first side and a upper fixing hole 332 at the free end of said second side. Said guiding holes 331 are dimensioned so that the screws 41 can pass through them.

The stem 32 is insertable along the body C of the humerus O, through the proximal epiphysis P, and passes through said anchoring element 2.

To guide the surgeon in inserting screws and in the orientation of the stem 32, the long side of the fixing structure 33 is arranged parallel to the humerus O, externally to the arm, during the surgical intervention. Said fixing structure is fixed to said rod 32 by a screw 41 passing through said upper fixing hole 332 and engages by screwing with said threaded fixing hole 322. In this way, the piston rod 32 and the structure 33 are rigidly fixed each other, according to a predetermined position.

Subsequently, one or more screws 41 are inserted, by means of a tool U, through said guiding side holes 331, so that they arrange in said grooves 321, suitably oriented so that said screws 41 fix in the bone tissue, both externally and internally of the anchoring element 2, i.e. in the internal cavity 24.

Once said screws 41 are inserted, by unscrewing the screw 41 fixed to the threaded fixing hole 322, the stem 32, it is possible to remove the structure 33.

FIGS. 9 and 10 show a third embodiment of the prosthesis for osteosynthesis 1 according to the present invention.

In this case, the support means 3 comprise one or more rigid guides 34, having a curved shape and are provided with solid inner channel, having an inlet hole 341 and an outlet hole 342. Said rigid guides 34 include a flange 343, with which a threaded screw 41 can engage. The prosthesis for osteosynthesis 1 may also include rigid guides 34′, which provide a lateral opened portion.

Said rigid guides 34 or 34′, provided with internal channel, include, e.g. in correspondence to the end 341, a valve, possibly, capable of facilitating the injection, of biological material or medicine (e.g. “door” type valves) repeated over time.

When the anchoring element 2 is inserted in the proximal epiphysis P, under the head of the humerus O, said rigid guides 34 or 34′ can be inserted through the lateral surface of the body C of the humerus O, so as to fit in the anchoring element 2.

Said rigid guides 34 or 34′ can be fixed to the humerus O by means of a screw 41, which fits through the flange 343, engaging with the bone tissue of the body C of the humerus O.

Through the channel of said rigid guides 34 curative substances and/or bone cement, if necessary, can be injected inside the bone tissue and the anchoring element 2.

In addition, at the end of said guides 34 or 34′ provided with the inner channel, valves may be provided for, capable of facilitating the injection, repeated over time, of biological material or medicine (e.g. “door” type valves).

FIGS. 11 and 12 show a third embodiment of the prosthesis for osteosynthesis 1 according to the present invention. In this case, the support means 3 comprise a stem 35, insertable in a diaphyseal channel hollowed by the surgeon in the body of the humerus O.

Said positioning means 4 comprising a parrot's beak hook 42, intended to couple with said anchoring element 2, when the latter is inserted in the proximal epiphysis P, under the head of the humerus O and said stem 35 is inserted and arranged through said anchoring element 2.

Said hook 42 may also be separable and can be coupled with said stem 35. In this way, said hook 42 can be coupled with the stem 35 and can be decoupled from it and extracted with a specific mechanism, after the insertion of said stem 35.

In order to facilitate the coupling between said stem 35 and said anchoring element 2, it is also possible to use a wire made of metal (or other material), which, from the axis of the stem 35, passing through the anchoring element, allows the surgeon to approach said anchoring element and said stem 35 along the correct trajectory that leads to the insertion of the stem 35 itself in the correct housing at the base of the anchoring element 2.

Said stem 35 can be made of semi-elastic material, also of resorbable type, e.g. triphosphate calcium, a material commercially called PIC, etc.

The prosthesis for osteosynthesis 1 according to the present invention may also include additional elements, which improve the osteosynthesis.

In particular, in FIGS. 13-16 it is shown the installation of the prosthesis for osteosynthesis 1 in combination with a prosthetic structure 5, having a further rod 51, a hood or head 52 and a body 53.

Said further rod 51 is inserted within the body C or the diaphysis of the humerus O and has, superiorly, a fixing pin 511 and a interlocking or threaded cavity 513; and, on the lateral surface, a plurality of longitudinal reliefs 512, insertable in the bone tissue for a better fixing of the stem 51 itself.

Said hood or head 52, possibly has a concavity, adapted to contain at least partly the bone tissue of the proximal epiphysis P or of the humerus O. Said hood or head 52 also has a coupling ring 521 provided with a seat 522, which function will be better described in the following, and rotatably adjustable according to arrow A.

The body 53 connects said further stem 51 and said hood or head 52. Said body 53 comprises an upper portion 531, capable of coupling with said seat 522 of said coupling ring 521 of said hood or head 52, and a lower portion 532, oriented at an angle γ with respect to said upper portion 531 and forming with it a shaped profile having a recess 533. At the lower end of said lower portion 532 is provided a coupling 534, in which said fixing pin 511 of said stem 51 is insertable and removably couple able.

As it can be seen, when the prosthesis for osteosynthesis 1 is installed together with said prosthetic system 5, considering said fourth embodiment, the lower end of the stem 35 of the prosthesis for osteosynthesis 1 is get stuck or coupled by screwing to said interlocking or threaded cavity 513, so that the anchoring element 2 arranges, at least partially, within the recess 533. In addition, it is observed that the best fit of the prosthetic system 5 to the human humerus O provides that the opening angle γ is comprised preferably between 125° and 135°.

It is considered that the present prosthetic structure 5 is also applicable with other embodiments of the prosthesis for osteosynthesis 1 described above.

An advantage of the prosthesis for osteosynthesis 1 according to the present invention is that the system allows the regrowth of bone tissue within the anchoring element 2.

The present invention has been described for illustrative but not limitative purposes, according to its preferred embodiments, but it is to be understood that modifications and/or changes can be introduced by those skilled in the art without departing from the relevant scope as defined in the enclosed claims.

1. A Prosthesis for osteosynthesis of a bone segment, in particular humerus, comprising
at least one anchoring element, having an internal cavity,
supporting means, coupled to the bone tissue of said bone segment, and
positioning means of said supporting means with respect to said anchoring element, (2), said positioning means comprise one or more threaded screws,
wherein said at least one anchoring element has the shape of a polyhedron having two triangle-shaped bases,
wherein said at least one anchoring element has one or more surfaces openings or holes, communicating with said internal cavity,
said supporting means comprise a plate, substantially “Y” shaped or bat wings shaped, with two upper ends, said plate being provided with a plurality of threaded holes and being positionable on the side surface of said bone segment,
said one or more threaded screws are insertable into the holes of said plate, engaging with said holes by the respective thread or by fixing, at least one of said threaded screws inserting through said surface passage holes, by entering into said internal cavity and
said plate comprises one or more anchoring hooks on each of said upper ends.

2-4. (canceled)

5. The prosthesis for osteosynthesis according to claim 1,
in that wherein said bases are isosceles triangle shaped, said bases having a vertex angle (α) and a pair of equal angles (β), said bases having a plurality of oval holes, and
in that said polyhedron has:
two side edges, corresponding to said equal angles (β) of said bases;
an vertex edge corresponding to said vertex angle (α), lower than said two side edges;
two side surfaces each one bounded by said vertex edge (23″) and by a respective side edge (23′), said side surfaces having at least one passage hole (21′), preferably having an oval form; and
a side surface, corresponding to the side of said base opposite to said vertex angle (α), bounded by said side edges, said side surface being open.

6. The prosthesis for osteosynthesis according to 1, wherein said bases are equal and/or said vertex angle (α) is greater than 90°.

7. The prosthesis for osteosynthesis according to claim 1, characterized in that wherein said anchoring element is made of titanium and/or resorbable material and/or inert material and/or fabric resulting from treated bone.

8.-9 (canceled)

10. The prosthesis for osteosynthesis according to claim 1, wherein said plate is made of deformable material, so that it can adapt to the surface of said bone segment, even in the installation phase, such as malleable titanium, or the like.

11. The prosthesis for osteosynthesis according to claim 1,
wherein said supporting means comprise
a stem, inserted in said bone segment, in order to be placed through said anchoring element within said cavity, said stem having one or more grooves on the side surface and a fixing threaded hole, arranged at one end, and
a removable fixing structure, comprising an inverted—“L” shaped element, having a first side and second side arranged at about 90°, said first side having a plurality of guiding side through holes and an through upper hole, said fixing structure being positionable so that said first side is arranged parallel to said bone segment, and
wherein said fixing means comprise one or more threaded screws insertable through said guiding side holes, so that they arrange in said grooves, appropriately oriented, and a further screw, inserted in said upper hole, so as to engage with said fixing threaded hole of said stem.

12. The prosthesis for osteosynthesis according to claim 1
wherein said supporting means comprise
at least one rigid guide having a first and a second end and insertable through the lateral surface of said bone segment, and
a flange fixed to said first end,
said at least one rigid guide being insertable in the bone tissue, so that said second end is inserted through at least one of said passage holes of said anchoring element, so that at least one portion said at least one rigid guide is arranged in said internal cavity, and
said positioning means comprise at least one threaded screw, engageable by screwing with said flange and with the bone tissue of said bone segment.

13. The prosthesis for osteosynthesis according to claim 12, wherein said at least one rigid guide has a curved shape.

14. The prosthesis for osteosynthesis according to anyone of claim 12, wherein said at least one rigid guide has an internal channel, provided with an inlet hole and an outlet hole.

15. The prosthesis for osteosynthesis according to claim 14, wherein said at least one rigid guide comprises a valve.

16. The prosthesis for osteosynthesis according to claim 12, characterized in that said rigid guide has an open lateral portion.

17. The prosthesis for osteosynthesis according to claim 1,
wherein said supporting means comprise a stem, insertable into a channel of said diaphyseal bone segment and inserted and arranged through said anchoring element within said cavity, and
said positioning means comprise at least one hook arranged at one end of said stem, said hook being intended to couple with said anchoring element.

18. The prosthesis for osteosynthesis according to claim 17, wherein said at least one hook is coupled in a removable way by said stem.

19. The prosthesis for osteosynthesis according to claim 17,
wherein said prosthesis for osteosynthesis comprises a guide wire, made of metal or deformable material, insertable through said stem and said anchoring element, so as to approach said stem to said anchoring element.

20. The prosthesis for osteosynthesis according to claim 1, wherein said prosthesis for osteosynthesis comprises a prosthetic structure, in its turn comprising
a further stem, insertable within said bone segment, in particular within the diaphysis of the humerus, having an upper fastening pin and an interlocking or threaded cavity and, on the side surface, a plurality of longitudinal reliefs, insertable within the bone tissue of said bone segment,
a cup or head, intended to be arranged on the proximal epiphysis of said bone segment and comprising a coupling ring provided with a rotatably adjustable seat, and
a body, for connecting said further rod and said hood or head, said body comprising an upper portion, capable of coupling with said seat of said coupling ring of said hood or head, and a lower portion, oriented at an opening angle (γ) with respect to said upper portion and forming with it a shaped profile having a recess, in which said anchoring element is intended to be inserted, the lower end of said lower portion being provided with a coupling, in which said fastening pin of said stem is insertable and can be coupled in a removable way,
said supporting means, being removably coupled with said interlocking or threaded cavity.
